(19) United States
(12) Patent Application Publication
(10) Pub. No.: US 2003/0190641 A1
(43) Pub. Date: Oct. 9, 2003

(54) Live cell method for observing cellular processes

(75) Inventors: Run Yu, Los Angeles, CA (US); Shlomo Melmed, Los Angeles, CA (US)

Correspondence Address: Pillsbury Winthrop LLP, Intellectual Property Group, 725 South Figueroa Street, Suite 2800, Los Angeles, CA, 90017-5406, US

(73) Assignee: CEDARS-SINAI MEDICAL CENTER

(21) Appl. No.: 10/252,309

(22) Filed: Sep. 23, 2002

Related U.S. Application Data
(60) Provisional application No. 60/370,912, filed on Apr. 8, 2002.

(51) Int. Cl. C12Q 1/68; G01N 33/53; G01N 33/567; G01N 33/574

(52) U.S. Cl. 435/6; 435/7.2

(57) ABSTRACT

Methods for observing the activity of compounds, cells, and cell components during a cellular event are provided. Such live cell observation allows one to assess the function and impact of various compounds and other molecules upon the biochemical pathways responsible for the event. Embodiments of the present invention describe the live cell observation of compounds' effects on PTTG during mitosis; specifically, the ramifications that such effects may have on treating aneuploidy and/or cancer. Alternate embodiments of the present invention describe the combination of the live cell method with high throughput screening, to efficiently identify and examine compounds for a desirable effect on a cellular event, such as compounds that may be effective in the treatment of aneuploidy or cancer.

### RELATED APPLICATIONS

This application claims the benefit of priority under 35 U.S.C. §119 of provisional U.S. application Ser. No. 60/370,912, filed Apr. 8, 2002, the contents of which are hereby incorporated by reference.

### GOVERNMENT RIGHTS

The invention described herein arose in the course of or under Grant No. CA75979 between the National Institutes of Health and the Doris Factor Molecular Endocrinology Laboratory at Cedars-Sinai Medical Center.

### FIELD OF THE INVENTION

This invention relates to methods of reducing the incidence of aneuploidy in mammalian cells; particularly, by affecting the expression of pituitary tumor transforming gene (PTTG). The invention further relates to a live cell method for observing cellular events, such as mitosis.

### BACKGROUND OF THE INVENTION

Cancer remains one of the leading causes of death in the United States and around the world. Its origins are largely unclear, and a reliable cure that spans the wide array of cancer types eludes discovery even after decades of intensive study. Given the tremendous impact that this illness continues to have on the human population, research efforts persist in the search for new therapeutic treatment modalities. To that end, various aspects of cancer pathology are constantly being investigated and analyzed in hopes of achieving a more thorough understanding of the underlying disease condition, and eventually, a cure.

One ubiquitous feature of human solid tumors is a condition known as aneuploidy; an abnormal number of chromosomes or chromosome segments present within an individual cell. Aneuploidy can lead to genetic instability, and may also promote further aneuploidy upon subsequent cellular division. Multiple mechanisms are thought to be involved in causing aneuploidy, including the activity of oncogenes, inappropriate cyclin expression, telomere defects, and mutations of tumor suppression genes. More specifically, it is believed that the oncogenes myc and ras enhance inappropriate DNA synthesis during the cell cycle, while the altered functionality of tumor suppressor genes such as p53 and adenomatous polyposis coli (APC) cause mitotic disturbances.

Pituitary tumor transforming gene (PTTG) is another oncogene that has been proposed as linked to tumorigenesis, although its role in that process was heretofore unclear. PTTG is likely a mammalian securin; one of a class of compounds responsible for inhibiting the progression of the biomolecular pathway that results in cleavage of cohesin (the compound that binds sister chromatids to one another during mitosis). PTTG has been thought to induce aneuploidy by improperly inhibiting chromosome segregation, but this hypothesis has not been substantiated.

Elucidating and understanding the mechanisms by which genes or other molecular cell components operate is limited largely by researchers' inability to physically observe these mechanisms in action. Although techniques are known for studying live cells and the processes occurring therein, the cells used in conjunction with such methods generally do not survive long enough for one to observe a complete cellular process, such as mitosis. Cell survival during such observation is dependent upon the maintenance of an environment that closely mimics the conditions of the cells' natural environment (i.e., mammalian internal body conditions). In general, when observing mammalian cells in this fashion, one must account for such factors as temperature, carbon dioxide (CO₂) concentration, pH, and environmental toxicity. CO₂ concentration and pH may be controlled by regulated perfusion in an observation system, and toxicity may be similarly regulated by providing an artificial environment compatible with the specimen; the environment presumably being non-toxic and biologically inert. Surprisingly, temperature, which is a critical aspect in the study of many biological systems, is often a difficult factor to control. This is due, at least in part, to the configurations of microscope apparatuses commonly used to perform live cell observation; often they can act as a heat sink, resulting in temperature gradients through a medium in which cells are disposed. However, products are available to aid researchers in addressing this problem. Still, even the satisfactory regulation of the above-enumerated factors is insufficient to maintain the cells in most live cell methods long enough to provide for the observation of various cellular processes.

An additional limitation of conventional live cell observation methods is the inability to track either a particular cell over a period of time (e.g., a stem cell as it differentiates), or only those cells embodying a particular characteristic (e.g., only those cells expressing a certain gene). In general, live cell observation is performed by studying multiple cells in a batch. Some, or even most of the cells may exhibit the particular characteristic being studied, but it is often difficult to create a batch of cells in which each and every cell exhibits the particular characteristic. This may lead to a significant amount of “noise” in study results, as the desired cells cannot be easily singled out.

It may be difficult to study any one, particular cell (or only those cells with a particular characteristic) because cells tend to migrate during a given study period, or because microscopic observation alone is insufficient to enable one to distinguish among different cells in a batch. Therefore, one may not be able to observe the same cell or cells at different points in time. These limitations result in research data being generated based on the behavioral trends of a batch of cells, rather than the actual behavior of individual cells or those exhibiting the studied characteristic; the latter, actual data enabling more definitive, substantiated results (i.e., with less study noise).

With the expansion of scientific knowledge regarding the human genome and the modern understanding of cellular genetics, there exists a need in the art for a method to observe longer duration, live cellular processes. Such an observation methodology would allow researchers to observe the interaction of various cellular components and processes with, for example, foreign compounds introduced to the cells or particular genes or gene products. This methodology may have important implications in the study and treatment of cancer, when coupled with the findings described herein with respect to PTTG.

### SUMMARY OF THE INVENTION

It is an object of an embodiment of the present invention to provide a method for observing the cellular activity of compounds, cells, and cell components. In accordance with alternate embodiments of the present invention, compounds, cells, and cell components may be viewed in conjunction with an observable cellular event; thereby, their function and impact with respect to that event may be assessed. For example, methods of the present invention describe diagnostic and other testing mechanisms by which putative proteins and other compounds may be examined for an effect on mitosis owing to their effect on the biological activity of PTTG or the biochemical pathways in which PTTG plays a role; specifically, mitosis. Alternate embodiments of the present invention describe methods of observing other cellular processes, as well.

It is yet another object of an embodiment of the present invention to provide methods for reducing the incidence of aneuploidy in a mammal; particularly by inhibiting the biological activity of PTTG, as, for example, by inhibiting its expression or signaling. It is a further object of the invention to provide methods of treating those diseases in which inhibiting the biological activity of PTTG would have a beneficial effect. Such diseases include, for example, various forms of cancer and other conditions that involve aneuploidy.

Other features and advantages of the invention will become apparent from the following detailed description, which illustrates, by way of example, various embodiments of the present invention.

### BRIEF DESCRIPTION OF THE DRAWINGS

FIG. 1 depicts the expression of enhanced green fluorescent protein tagged PTTG (PTTG-EGFP) in accordance with an embodiment of the present invention. The results of Western blotting of H1299 cells transfected with EGFP or PTTG-EGFP are indicated.

FIG. 2 depicts chromosomal localization of PTTG-EGFP in accordance with an embodiment of the present invention. H1299 cells were transfected with PTTG-EGFP, p55CDC-EGPF or EGFP alone, and then treated hypotonically, spun onto chamber slides and fixed and stained with human anti-centromere and Hoechst 33342. EGFP alone did not associate with chromosomes. FIGS. 2A and 2D illustrate chromosomes; FIG. 2B illustrates PTTG-EGFP; FIGS. 2C and 2F illustrate centromeres; and FIG. 2E illustrates p55CDC-EGFP.

FIG. 3 depicts PTTG-EGFP degradation and the anaphase bridge in PTTG-EGFP-expressing cells in accordance with an embodiment of the present invention. Single live cells expressing PTTG-EGFP were continuously observed and representative images are shown. FIG. 3A illustrates PTTG degradation before anaphase onset. FIG. 3B illustrates a persistent anaphase bridge resulting in aborted cytokinesis (arrow indicates anaphase bridge). The time at which each image was taken is included with each individual frame.

FIG. 4 depicts chromosome non-segregation and aneuploidy resulting from failure of PTTG-EGFP degradation in accordance with an embodiment of the present invention. FIG. 4A illustrates the absence of chromosome segregation with completed cytokinesis (arrow indicates non-segregated chromosomes). FIG. 4B illustrates incomplete chromosome segregation with aborted cytokinesis (asterisk indicates a micronucleus; D2 indicates second day of observation). FIG. 4C illustrates a cell with doubled nuclear size as a result of chromosome non-segregation.

FIG. 5 depicts chromosome non-segregation and aneuploidy in cells expressing non-degradable mutant PTTG-EGFP (DM-PTTG-EGFP) in accordance with an embodiment of the present invention. FIG. 5A illustrates chromosome non-segregation and cytokinesis in a live cell expressing DM-PTTG-EGFP (arrow indicates non-segregated chromosomes). FIGS. 5B-5F illustrate that cells expressing DM-PTTG-EGFP were fixed; mitotic spindles (FIGS. 5B-5E) and centrosomes (FIG. 5F) being stained with an antibody to α- or γ-tubulin. Cells were also stained for actin, and DNA stained by Hoechst 33342. The cell depicted in FIG. 5B was at metaphase; 5C at early cytokinesis; 5D, 5F, and 5G at late cytokinesis; and cell 5E post cytokinesis. The cells depicted in FIGS. 5B through 5E corresponded roughly to the first four frames depicted in FIG. 5A.

FIG. 6 depicts tabular data relating to mitosis of cells expressing PTTG-EGFP in accordance with an embodiment of the present invention. Mitosis of single, live H1299 cells untransfected (Control), expressing EGFP only (EGFP), expressing PTTG-EGFP (PTTG-EGFP), or expressing non-degradable mutant PTTG-EGFP (DM-PTTG-EGFP) was observed for the presence (y) or absence (n) of PTTG degradation, chromosome segregation and condensation, and cytokinesis. “y/n” represents incomplete chromosome segregation or cytokinesis, and “*” represents the number of anaphase bridges. “M” or “P” represents chromosome decondensation at metaphase or prophase. “Macro” and “micro” represent macronucleus and micronucleus, respectively.

### DETAILED DESCRIPTION OF THE INVENTION

The present invention is based on the discovery of a live cell testing method that may be used to observe cellular processes and the effects that various compounds have on those processes. Surprisingly, the live cell testing method of the present invention may be used to observe cellular events over a substantial period of time, allowing an observer to view entire processes or events, such as a complete iteration of cell division. It is believed that the longevity of the methods of the present invention are due, at least in part, to the capacity of the preferred cell lines used in accordance therewith to remain viable outside the body; although the methods described herein are in no way limited exclusively to those cell lines, especially where a cell process one wishes to observe is brief in duration.

In a preferred embodiment, human H1299 cells, a human non-small cell lung carcinoma cell line, may be used in accordance with the methods of the present invention. The inventors have surprisingly identified human H1299 as a cell line that remains viable for a longer period of time in live cell observation when compared to cell lines used in other, conventional methods. However, other cell lines may be used in conjunction with the various embodiments of the present invention, especially in those instances where cell processes under observation are brief. Such other cell lines may include, but are in no way limited to, JEG3, AtT20 (a mouse pituitary tumor cell line), and mouse embryonic fibroblasts.

Cellular processes suitable for observation in accordance with the methods of the present invention may include both “directly observable processes” and “indirectly observable processes.” For purposes of the present invention, directly observable processes include any cellular event or series of events that manifests in a physical change to the cell structure, its contents, or its physical surroundings. Examples of directly observable processes may include, but are in no way limited to, mitosis and the individual stages thereof (e.g., interphase, prophase, metaphase, anaphase, telophase), apoptosis, necrosis, or processes that affect the cell membrane or cell movement. Directly observable processes include those in which a change occurs to the configuration or structure of a cell or its components over a period of time, as would be visually recognized by one of skill in the art.

For purposes of the present invention, indirectly observable processes include any cellular event or series of events that does not manifest in a visually cognizable change to the physical cell structure. Instead, such processes may include, but are in no way limited to, those in which a compound is generated (e.g., a hormone), or digested or otherwise eliminated from the cell or its surroundings (e.g., a cell nutrient, such as glucose). Such indirectly observable processes may be viewed quantitatively, such as by examining the extent to which a compound is present in or around the cell under observation. These processes are generally difficult or impossible to observe with the aid of magnification alone, as they do not effect an easily visible, physical change to the cell or its components. The various tagging methods discussed below may be particularly advantageous when studying indirectly observable processes in accordance with embodiments of the present invention; although these tagging methods may be used to study directly observable processes, as well.

In one preferred embodiment of the present invention, the observed cellular process is mitosis, or cell division. Although the complex nature of a process such as mitosis lends itself to the examination of the behavior of a wide array of compounds and numerous cellular components during the course thereof, in a preferred embodiment of the present invention, mitosis is examined in conjunction with PTTG or items that may affect the same. Such PTTG-affecting items may include, but are in no way limited to, molecules, compounds, proteins, hormones, vaccines, therapeutic agents, pharmaceuticals, combinations thereof, and any other item that may affect PTTG or the role it plays in a cellular process, such as, by way of example, mitosis or tumorigenesis.

It is believed that PTTG plays a role in the progression (or hindrance) of mitosis, and it is further believed that improper cell signaling or increased amounts of PTTG may lead to aneuploidy under certain conditions. It may therefore be advantageous to examine the effects of various compounds with respect to the role PTTG plays in mitosis, or the effect that such compounds may have on PTTG itself. The results of such studies may be the experimental precursors for therapeutic compounds useful in the treatment of, for example, cancer and other disease conditions involving aneuploidy.

Additional embodiments of the present invention incorporate fluorescent or other tagging techniques, such that a specific compound, cell, or cell component (e.g., an organelle) may be observed in conjunction with the live cell testing method during a directly or indirectly observable process. In one embodiment of the present invention, one uses a fluorescent marker, such as enhanced green fluorescent protein (EGFP) to “tag” a compound or cell component for observation during the progression of a particular cellular process. Other suitable fluorescent or non-fluorescent markers and yet further tagging techniques will be readily apparent to one of skill in the art; appropriate markers can be selected by one of such skill without undue experimentation, as can a suitable technique for using them. For example, other suitable fluorescent markers may include enhanced yellow fluorescent protein, red fluorescent protein, rhodamine, fluoresceine, and cy5, while other suitable non-fluorescent markers may include horseradish peroxidase, epitope tags, and gold particles. The selection of suitable markers may depend, at least in part, on the characteristics of the compound, cell, or cell component sought to be tagged. Any conventional tagging technique may be used in accordance with various embodiments of the present invention, including, but in no way limited to, chemical reaction and noncovalent conjugating.

In a preferred embodiment of the present invention, PTTG may be tagged with EGFP, and its role observed during the course of mitosis. The observation of this role may include examining the interaction of PTTG with other compounds or cell components during the progression of cell division (i.e., directly observable processes), or it may include examining the digestion or generation of particular compounds during the course thereof (i.e., indirectly observable processes).

It may be desirable to observe multiple compounds, cells, or cellular components during the course of a cellular process. Or, it may be desirable to tag both a compound and a particular cell to observe, for example, the uptake of the compound by the cell.

Thus, in an alternate embodiment of the present invention, one may tag multiple compounds, cells, cell components, or a combination thereof with an appropriate number of different markers. In preferred such embodiments, different items may be tagged with different markers (e.g., those displaying different colors), such that the distinction among the various items may be readily, visually ascertained when the markers fluoresce. However, there may be instances where one does not desire to distinguish among such items, and in such cases it may be desirable to employ the same marker for multiple compounds, cells, cell components, or combinations of the same.

In a further embodiment of the present invention, the live cell testing method may be used in combination with high throughput screening; providing a method for both identifying and testing compounds for a desired effect on a cellular process. Such a method may enhance the efficiency by which researchers are able to find and examine the efficacy of potentially therapeutic compounds. High throughput screening is a process in which a number of compounds are tested for binding or other biological activity with respect to target molecules, and assays and related devices and laboratory materials are available from a number of providers. For instance, Perkin Elmer, Inc. manufactures high throughput assay platforms useful in this process.

The compounds studied with high throughput screening may include, for example, enzymatic inhibitors (e.g., competitors for a natural ligand to a receptor), or may be agonists or antagonists for receptor-mediated intracellular processes. An advantage of high throughput screening is the rapidity with which large numbers of compounds can be examined for reactivity with the target. Also significant is the fact that high throughput screening has been developed into an automated process, enhancing process efficiency while reducing both labor requirements and the opportunity for human error. Currently, various companies in the pharmaceutical industry utilize high throughput screening to identify new drugs, and some biotechnology companies utilize high throughput screening to determine the function of biomolecules, such as proteins.

The use of high-throughput screening in conjunction with live cell observation may provide for rapid detection of effective compounds in the treatment of cancer and other diseases. In one preferred embodiment of the present invention, the combination of high throughput screening with the live cell method may provide for further study of PTTG and the role it plays in mitosis as compounds screened for reactivity with PTTG are subsequently studied in live cell observation of mitosis. Live cell observation of mitosis generally takes less than about 24 hours, and high throughput screening may be employed to rapidly identify target compounds. Therefore, the combination of these techniques may provide a method for efficiently identifying and observing proteins that, for example, interact with components of the PTTG cell signaling cascade, effect PTTG over-expression, or degrade PTTG protein products before anaphase.

### EXAMPLES

These Examples demonstrate that PTTG causes aneuploidy in single, live human cells by disrupting mitosis. Based on the proposed PTTG securin function, it has been hypothesized that PTTG may disturb mitosis, but this hypothesis has not been substantiated. The ensuing Examples employ the live cell method of the present invention to demonstrate that PTTG indeed disrupts mitosis and causes aneuploidy.

This study further establishes that PTTG is, in fact, a mammalian securin, based on the following lines of evidence, observed through the live cell method of the present invention: PTTG localizes to mitotic chromosomes and is degraded shortly before the onset of anaphase, and PTTG over-expression inhibits chromosome segregation. Absence of, or incomplete PTTG degradation is a critical step in aneuploidy induction because even cells expressing medium levels of PTTG still give rise to normal daughter cells, as long as PTTG is degraded. The importance of PTTG degradation is dramatically illustrated in that all cells expressing the non-degradable mutant undergo abnormal mitosis and exhibit aneuploidy. During tumorigenesis, PTTG overexpression may result in incomplete degradation, causing abnormal mitosis and aneuploidy.

Aneuploidy is one of the hallmarks of tumors. Although multiple mechanisms may cause aneuploidy, it has not previously been demonstrated how a specific aneuploidy is produced in the tumorigenesis process. The live cell method of the present invention has allowed in the inventors to demonstrate that PTTG directly causes chromosome copy doubling. Since the examined cells only expressed PTTG for a matter of hours, the resultant aneuploidy is likely a direct consequence of PTTG expression. In previous aneuploidy studies, using stable oncogene transfectants, or tumor suppressor gene-deficient mice, aneuploidy was generated weeks or even months after generations of cell division. It is unclear whether aneuploidy observed in those studies occurred directly or indirectly as a consequence of genetic manipulations. However, the continuous observation of the same cells both before and after mitosis performed herein confirmed that the aneuploid cells observed were indeed normal prior to the experiment. Absent the live cell method of the present invention, such an observation may not have been made; and, indeed, was not made.

In summary, the ensuing Examples demonstrate that PTTG disrupts mitosis and causes aneuploidy in single, live human cells due to failure of PTTG degradation as a result of overexpression. The results provide direct evidence of transformation from a normal to an aneuploid mammalian cell by an oncogene. Moreover, the Examples demonstrate the effect of the live cell method of the present invention in substantiating scientific hypotheses that would otherwise have been impossible to substantiate and would remain grounded largely in conjecture.

### Example 1

### Cells and Plasmids

Human H1299 cells were grown in Dulbeccos's Modified Eagle Medium (DMEM) with 10% Fetal Bovine Serum (FBS) and transfected with Lipofectamine 2000 (obtained from Invitrogen Corporation; Carlsbad, Calif.; hereinafter “Invitrogen”). PTTG-EGFP and p55CDC-EGFP were constructed by cloning PTTG or p55CDC (obtained from Amgen, Inc.; Thousand Oaks, Calif.) into pEGFP-N3 (obtained from BD Biosciences Clontech; Palo Alto, Calif.; hereinafter “Clontech”). Non-degradable mutant PTTG-EGFP was obtained from Dr. Chris McCabe (University of Birmingham, England, UK). EGFP was at the C-terminus of PTTG or p55CDC. Cells were studied (microscopy or by Western blot) 18 to 24 hours after transfection.

### Example 2

### Immnuofluorescent Staining

H1299 cells transfected with PTTG-EGFP, p55CDC-EGFP, or EGFP were trysinized, washed with DMEM and resuspended in hypotonic buffer (10 mM Tris, 10 mM NaCl, 5 mM MgCl₂, pH 7.0) for 15 minutes, spun onto a Nunc chamber slide at 1,350 g for 3 minutes, and immediately fixed with ice-cold ethanol. Cells were rehydrated and stained with human anti-centromere serum (obtained from Rheumatology Diagnostics Laboratory, Inc.; Los Angeles, Calif.) and anti-human rhodamine, counterstained with Hoechst 33342, and observed with appropriate filters. Staining of γ-tubulin of cells grown on coverslips was performed as described in R. Yu et al., “Pituitary Tumor Transforming Gene (PTTG) regulates placental JEG-3 cell division and survival: evidence from live cell imaging,” Mol. Endocrinol. 14:1137-1146 (2000). Cells were fixed in methanol for staining with antibodies to γ-tubulin and actin (obtained from Sigma-Aldrich, Inc.; St. Louis, Mo.) and rhodamine-labeled second antibodies used.

### Example 3

### Single, Live Cell Imaging

Observation of individual live cells over 48 hours or longer was performed by incubating cells in an FCS2 Closed Perfusion System (obtained from Bioptechs; Butler, Pa.). Cells were perfused with CO₂-independent L15 medium (obtained from Invitrogen) supplemented with 10% FBS and penicillin/streptomycin and saturated with ambient air. Perfusion chamber temperature was set to 37° C., and cells were grown in the perfusion system for up to a week until confluency.

The perfusion chamber was placed on an inverted fluorescence microscope (obtained from Nikon Corporation; Melville, N.Y.) and observed with a 40× extra-long working distance objective lens. Cells were observed from every few seconds to every several hours depending on the speed of cell changes. Durations of mitosis phases were determined by counting the minutes between two sequential mitotic milestones. Phase-contrast and EGFP fluorescent images were taken simultaneously at frequencies ranging from every minute (e.g., during metaphase to anaphase transition) to every few hours (e.g., after telophase), with a CCD digital camera.

Relative fluorescence intensity was objectively determined with the application of two neutral density filters (NDFs). Each NDF reduces incident light by 50%. High fluorescence was defined when a cell was clearly visualized after application of two NDFs. Medium fluorescence was defined as a cell clearly visualized after application of one but not two NDFs. Low fluorescence was defined as a cell only visualized when neither NDF was applied.

### Example 4

### PTTG Localizes to Mitotic Chromosomes

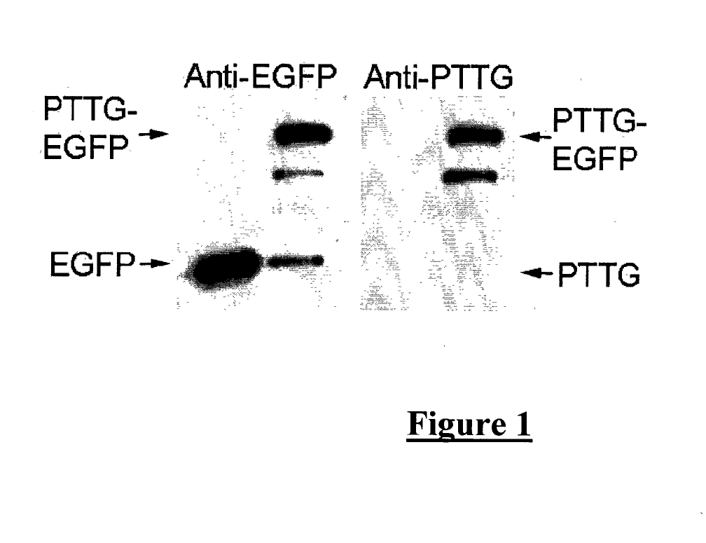

To directly test whether PTTG causes aneuploidy, an EGFP-tagged PTTG (PTTG-EGFP) was expressed in human H1299 cells and mitosis of individual live cells expressing PTTG-EGFP was observed. H1299 cells transfected with EGFP or PTTG-EGFP were lysed in SDS-PAGE lysis buffer 24 hours after transfection, and equal amounts of cell lysates subjected to Western blotting (FIG. 1). The membrane was first blotted with mouse anti-EGFP (obtained from Clontech) and anti-mouse peroxidase; washed in 0.3% NaN₃, reblotted with rabbit anti-PTTG (obtained from Zymed Laboratories, Inc.; South San Francisco, Calif.) and anti-rabbit peroxidase; and developed with ECL (available from Amersham Biosciences, Inc.; Piscataway, N.J.). For chromosomal localization of PTTG-EGFP, H1299 cells transfected with PTTG-EGFP, p55CDC-EGFP or EGFP were treated hypotonically and spun onto chamber slides, fixed and stained with human anti-centromere and Hoechst 33342 (available from Aventis Pharmaceuticals, Inc.; Bridgewater, N.J.). EGFP alone did not associate with chromosomes. Endogenous PTTG levels were undetectable in H1299 cells. PTTG-EGFP was reactive to antibodies against both PTTG and EGFP and subcellular PTTG-EGFP localization was similar to that of PTTG.

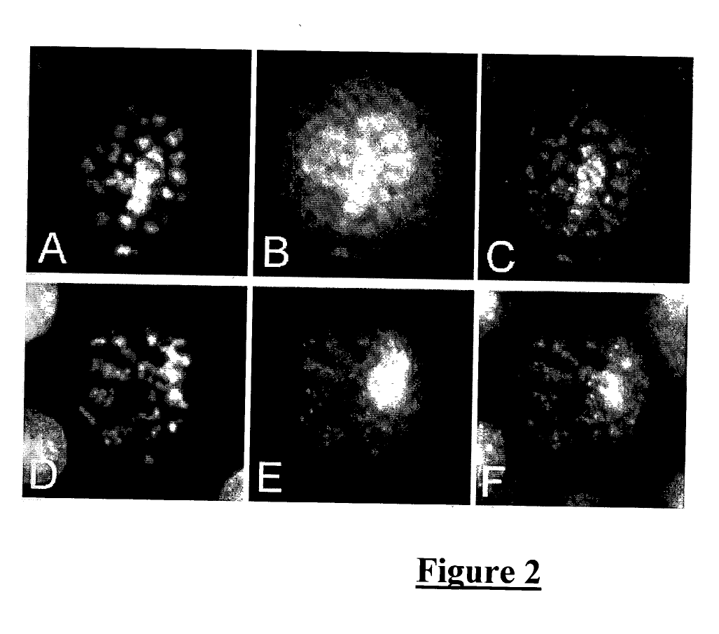

Since cohesin is localized to chromosomes, and separin and PTTG should be close to cohesin, PTTG may also localize to chromosomes. In previous experiments, mitotic spindle-associated PTTG was predominant and it was not possible to ascertain whether PTTG also localizes to mitotic chromosomes (not shown). Cytosolic proteins were therefore removed and significant PTTG-EGFP chromosomal localization was observed (FIG. 2). Unlike p55CDC which co-localizes with centromeres, PTTG-EGFP distributed evenly on mitotic chromosomes. It was also evident that H1299 cell chromosomes harbor a single centromere (FIGS. 2C and 2F).

### Example 5

### PTTG Overexpression Blocks Mitosis Progression

As depicted in Table 1, prophase (Pro), metaphase (Meta), anaphase (Ana), and telophase (Telo) durations were recorded (in minutes) in untransfected control cells (Control), cells expressing EGFP only (EGFP), and in cells expressing PTTG-EGFP (PTTG-EGFP) 18 hours after transfection. EGFP alone, or low levels of PTTG-EGFP, did not affect the duration of each phase, but medium or high PTTG-EGFP levels dramatically prolonged prophase and metaphase, indicating that PTTG blocks the progression of mitosis to anaphase. For some cells expressing PTTG-EGFP, no distinct anaphase or telophase was seen because of abnormal chromosome segregation and cytokinesis.

TABLE 1
Duration of Mitosis Phases of Cells Expressing PTTG-EGFP

| | Pro | Meta | Ana | Telo |
|---|---|---|---|---|
| Control (n = 16) | 15 ± 2 | 25 ± 3 | 4 ± 1 | 5 ± 0 |
| EGFP (n = 28) | 14 ± 1 | 16 ± 2 | 4 ± 0 | 6 ± 1 |
| PTTG-EGFP Low (n = 11) | 25 ± 3 | 26 ± 4 | 4 ± 0 | 5 ± 0 |
| PTTG-EGFP Medium (n = 10) | 35 ± 12* | 49 ± 16* | 4 ± 0 | 5 ± 0 |
| PTTG-EGFP High (n = 3) | 59 ± 7* | 112 ± 31* | N/A | N/A |

*p < 0.01 compared to Control or EGFP

### Example 6

### PTTG Overexpression Disrupts Mitosis and Causes Aneuploidy

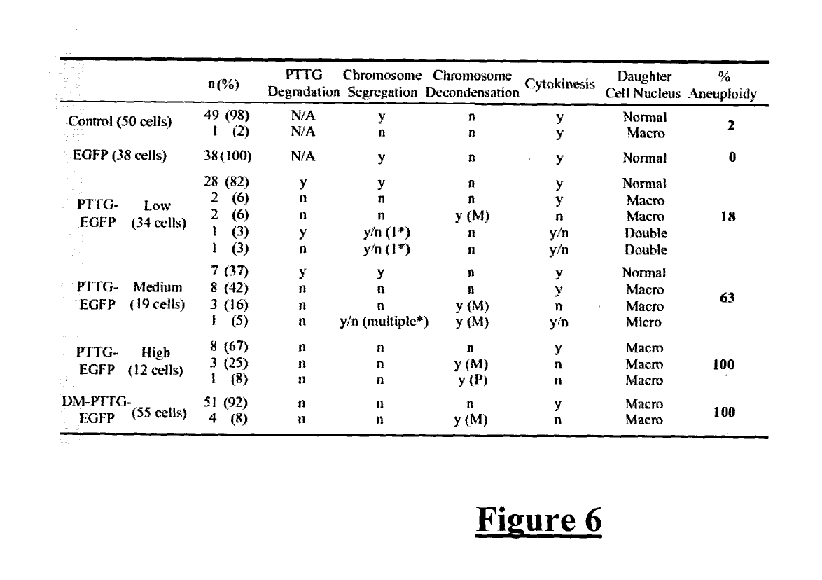

As depicted in FIG. 6, the destiny of mitosis (from G2, prophase, or metaphase to the subsequent interphase) was observed in 50 untransfected cells, 38 cells expressing EGFP only, and 65 cells expressing PTTG-EGFP. All but one untransfected cell, and all cells expressing EGFP alone exhibited appropriate chromosome segregation and cytokinesis, resulting in two normal daughter cells. EGFP expression levels did not affect the mitosis outcome of cells expressing EGFP alone (data not shown).

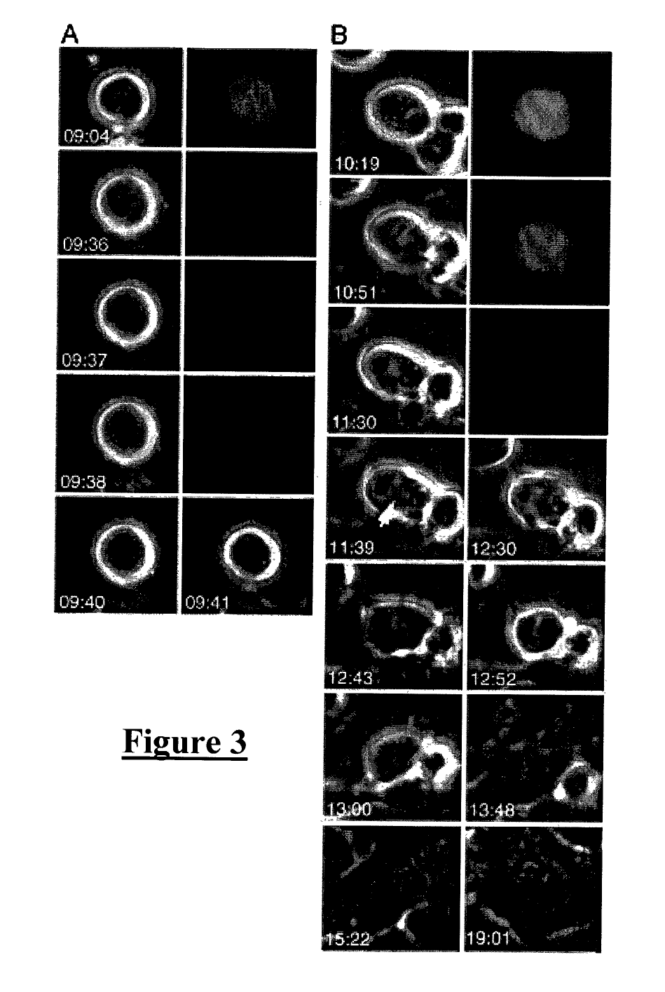

In all PTTG-EGFP-expressing cells that underwent apparently normal mitosis (i.e., normal chromosome segregation, no chromosome decondensation, and normal cytokinesis), PTTG-EGFP was degraded about 1 minute prior to the onset of anaphase (FIG. 6; FIG. 3A), consistent with the securin function of PTTG. EGFP was stable during and after mitosis, and p55CDC-EGFP was stable throughout mitosis but degraded early in G1 (data not shown). An anaphase bridge was infrequently observed (2/65 cells), and persisted for more than one hour, resulting in aborted cytokinesis and a “daughter” cell with two nuclei (FIG. 6; FIG. 3B).

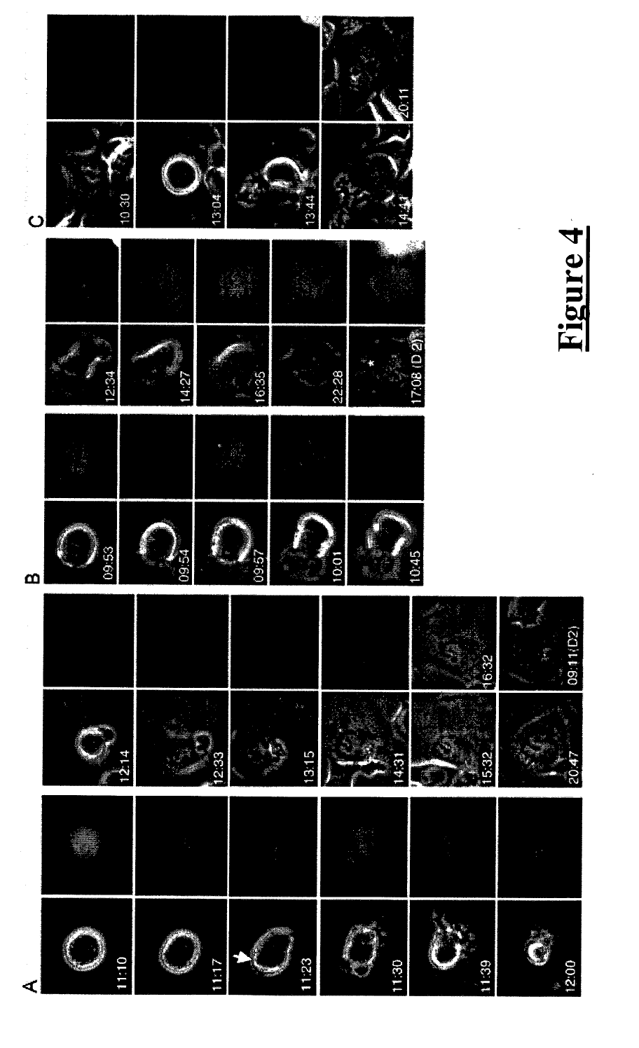

Failure of PTTG degradation is associated with chromosome non-segregation (FIG. 6); cytokinesis, however, occurred independently of chromosome segregation (FIG. 6; FIG. 4A). In this asymmetrical cytokinesis, metaphase chromosomes first moved closer to one cell pole, followed by cell elongation and appearance of a cell midline furrow. Complete non-segregation during cytokinesis resulted in one daughter cell containing all chromosomes, turning into a cell harboring a macronucleus, and the other non-viable cell devoid of a nucleus (FIG. 4A).

Asymmetrical cytokinesis without chromosome segregation was the feature of abnormal mitosis most commonly observed (FIG. 6). Segregation was sometimes incomplete, with multiple anaphase bridges and the appearance of several micronuclei (FIG. 4B). To demonstrate that the metaphase cells previously underwent a normal interphase, a PTTG-EGFP-expressing cell was shown to progress from interphase, mitosis, to interphase again but doubled its nuclear size due to incomplete PTTG-EGFP degradation and consequent chromosome non-segregation (FIG. 4C). In a few cells, chromosomes were decondensed after extended prophase or metaphase, and no cytokinesis was observed, resulting in a cell containing a macronucleus (FIG. 6). Chromosome decondensation mostly occurred at metaphase and occasionally at prophase. In both cases, PTTG-EGFP degraded continuously but complete degradation was only achieved after chromosome decondensation.

### Example 7

### A Non-Degradable Mutant PTTG Invariably Causes Aneuploidy

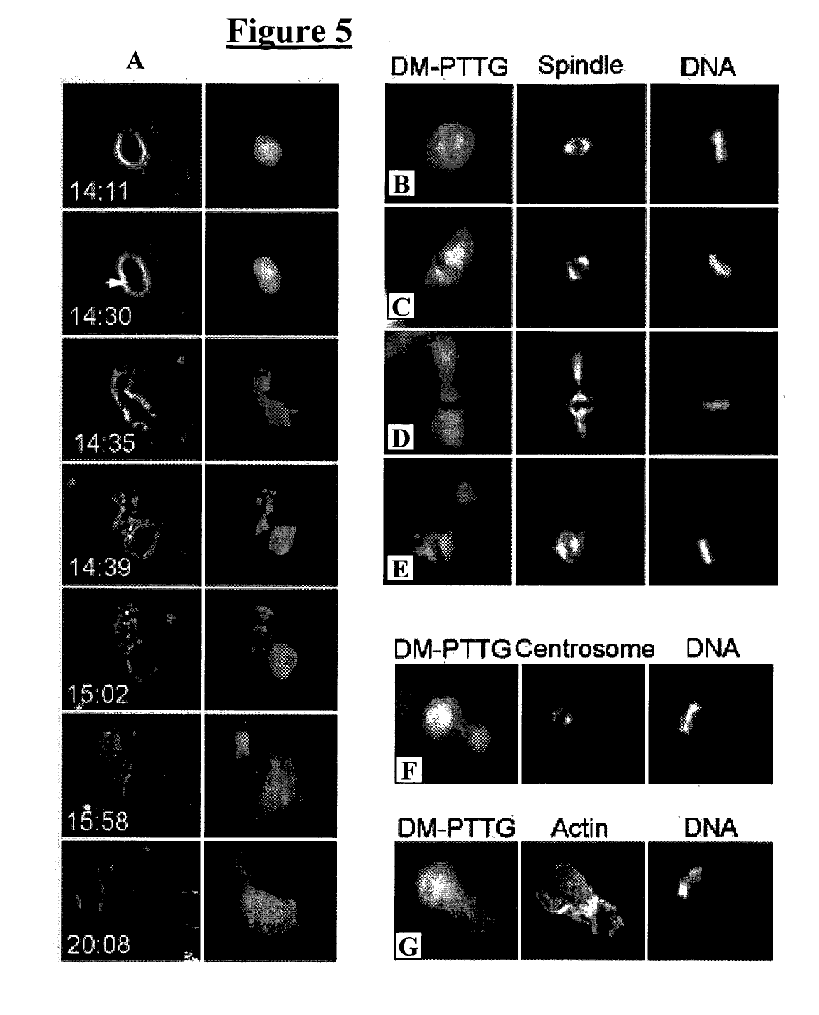

As failure of PTTG degradation is associated with chromosome non-segregation, an EGFP-tagged non-degradable mutant PTTG (DM-PTTG-EGFP) was expressed in H1299 cells and chromosome segregation and cytokinesis of individual live cells observed (FIG. 6; FIG. 5A). Similar to the wild-type PTTG, DM-PTTG localizes to mitotic spindles (FIGS. 5B-5G) and chromosomes (data not shown). All 55 cells observed expressing non-degradable PTTG exhibited abnormal mitosis, irrespective of expression levels, and none of the cells degraded mutant PTTG-EGFP or segregated chromosomes. Most (51/55) cells underwent asymmetrical cytokinesis, while some cells decondensed chromosomes; macronuclei ensued in both cases. During asymmetrical cytokinesis, chromosomes and mitotic spindles were closely attached and moved to one cell pole (FIGS. 5B-5G). The cytokinesis furrow occurred roughly at the position of the distal centrosome (FIGS. 5C and 5D). Both centrosomes were retained in the daughter cell containing chromosomes (FIGS. 5E and 5F). Actin was concentrated between the two daughter cells (FIG. 5G), suggesting a normal actomyosin mechanism in the cytokinesis.

Macronuclei, micronuclei, and multiple nuclei are signs of aneuploidy. Aneuploidy correlated with PTTG-EGFP expression levels (FIG. 6), and only occurred in 0-2% of daughter cells if parent cells were untransfected or expressed EGFP alone. However, this rate increased to 18% or 63% if parent cells expressed low or medium levels of PTTG-EGFP, respectively. All daughter cells derived from parent cells expressing high levels of PTTG-EGFP were aneuploid.

While the description above refers to particular embodiments of the present invention, it will be understood that many modifications may be made without departing from the spirit thereof. For instance, the protease inhibitors of the present invention may be used in the treatment of any number of conditions where inflammation is observed, as would be readily recognized by one skilled in the art and without undue experimentation. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

We claim:

1. A method of studying a cellular process in a live cell, comprising:
including at least one human H1299 cell in a media; and
observing the cellular process.

2. The method of claim 1, further comprising tagging an observable item with a marker.

3. The method of claim 2, wherein the observable item is selected from the group consisting of a compound, a cellular component, and a cell.

4. The method of claim 2, wherein the observable item is pituitary tumor transforming gene (PTTG).

5. The method of claim 2, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

6. The method of claim 2, wherein tagging an observable item with a marker further comprises tagging multiple observable items with either the same marker or different markers.

7. The method of claim 1, wherein the cellular process is selected from the group consisting of a directly observable cellular process and an indirectly observable cellular process.

8. The method of claim 1, wherein the cellular process is mitosis.

9. The method of claim 8, wherein observing the cellular process further comprises observing an interaction of pituitary tumor transforming gene (PTTG) with a test item.

10. The method of claim 9, wherein the test item is selected from the group consisting of a molecule, a compound, a protein, a hormone, a vaccine, a therapeutic agent, a pharmaceutical, an item that affects PTTG or the role it plays in a cellular process, and combinations thereof.

11. The method of claim 9, further comprising tagging the test item with a marker.

12. The method of claim 11, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

13. The method of claim 1, further comprising setting the temperature of the media.

14. The method of claim 13, wherein setting the temperature of the media further comprises setting the temperature of the media at about 37° C.

15. The method of claim 1, further comprising setting the carbon dioxide (CO₂) concentration of the media.

16. The method of claim 15, wherein setting the CO₂ concentration of the media further comprises setting the CO₂ concentration of the media within a range of from about 4% to about 6%.

17. The method of claim 1, further comprising saturating the media with ambient air.

18. The method of claim 1, further comprising including an antibiotic in the media.

19. The method of claim 1, wherein the cellular process is stem cell differentiation.

20. The method of claim 2, wherein the observable item is identified with high throughput screening.

21. The method of claim 9, wherein the test item is identified with high throughput screening.

22. A method of studying a cellular process in a live cell, comprising:
including at least one cell in a media;
observing the cellular process,
wherein the cell is selected from a cell line that remains alive for at least 24 hours when placed in the media.

23. The method of claim 22, further comprising tagging an observable item with a marker.

24. The method of claim 23, wherein the observable item is selected from the group consisting of a compound, a cellular component, and a cell.

25. The method of claim 23, wherein the observable item is pituitary tumor transforming gene (PTTG).

26. The method of claim 23, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

27. The method of claim 23, wherein tagging an observable item with a marker further comprises tagging multiple observable items with either the same marker or different markers.

28. The method of claim 22, wherein the cellular process is selected from the group consisting of a directly observable cellular process and an indirectly observable cellular process.

29. The method of claim 22, wherein the cellular process is mitosis.

30. The method of claim 29, wherein observing the cellular process further comprises observing an interaction of pituitary tumor transforming gene (PTTG) with a test item.

31. The method of claim 30, wherein the test item is selected from the group consisting of a molecule, a compound, a protein, a hormone, a vaccine, a therapeutic agent, a pharmaceutical, an item that affects PTTG or the role it plays in a cellular process, and combinations thereof.

32. The method of claim 30, further comprising tagging the test item with a marker.

33. The method of claim 32, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

34. The method of claim 22, further comprising setting the temperature of the media.

35. The method of claim 34, wherein setting the temperature of the media further comprises setting the temperature of the media at about 37° C.

36. The method of claim 22, further comprising setting the carbon dioxide (CO₂) concentration of the media.

37. The method of claim 36, wherein setting the CO₂ concentration of the media further comprises setting the CO₂ concentration of the media within a range of from about 4% to about 6%.

38. The method of claim 22, further comprising saturating the media with ambient air.

39. The method of claim 22, further comprising including an antibiotic in the media.

40. The method of claim 22, wherein the cellular process is stem cell differentiation.

41. The method of claim 22, wherein the cell line is human H1299.

42. The method of claim 23, wherein the observable item is identified with high throughput screening.

43. The method of claim 30, wherein the test item is identified with high throughput screening.

44. A method to study the efficacy of a test item, comprising:
identifying the test item with high throughput screening;
including at least one cell in a media;
introducing the test item to the media; and
observing a cellular process involving both the test item and the cell.

45. The method of claim 44, further comprising tagging an observable item with a marker.

46. The method of claim 45, wherein the observable item is selected from the group consisting of a compound, a cellular component, and a cell.

47. The method of claim 45, wherein the observable item is pituitary tumor transforming gene (PTTG).

48. The method of claim 45, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

49. The method of claim 45, wherein tagging an observable item with a marker further comprises tagging multiple observable items with either the same marker or different markers.

50. The method of claim 44, wherein the cellular process is selected from the group consisting of a directly observable cellular process and an indirectly observable cellular process.

51. The method of claim 44, wherein the cellular process is mitosis.

52. The method of claim 51, wherein observing the cellular process involving both the test item and the cell further comprises observing an interaction of pituitary tumor transforming gene (PTTG) with the test item.

53. The method of claim 44, wherein the test item is selected from the group consisting of a molecule, a compound, a protein, a hormone, a vaccine, a therapeutic agent, a pharmaceutical, an item that affects PTTG or the role it plays in a cellular process, and combinations thereof.

54. The method of claim 44, further comprising tagging the test item with a marker.

55. The method of claim 54, wherein the marker is selected from the group consisting of enhanced green fluorescent protein (EGFP), enhanced yellow fluorescent protein, red fluorescent protein, rhodamine, fluoresceine, cy5, horseradish peroxidase, epitope tags, and gold particles.

56. The method of claim 44, further comprising setting the temperature of the media.

57. The method of claim 56, where in setting the temperature of the media further comprises setting the temperature of the media at about 37° C.

58. The method of claim 44, further comprising setting the carbon dioxide (CO₂) concentration of the media.

59. The method of claim 58, wherein setting the CO₂ concentration of the media further comprises setting the CO₂ concentration of the media within a range of from about 4% to about 6%.

60. The method of claim 44, further comprising saturating the media with ambient air.

61. The method of claim 44, further comprising including an antibiotic in the media.

62. The method of claim 44, wherein the cellular process is stem cell differentiation.

63. The method of claim 44, wherein introducing the test item to the media further comprises introducing the test item to the cell.

64. The method of claim 44, wherein the cell is selected from a cell line that remains alive for at least 24 hours when placed in the media.

65. The method of claim 44, wherein the cell line is human H1299.
